(19) United States
(12) Patent Application Publication
(10) Pub. No.: US 2022/0002937 A1
(43) Pub. Date: Jan. 6, 2022

(54) METHOD FOR MAKING FIBER CONTAINING ACTIVE INGREDIENTS FROM CHINESE HERB

(71) Applicants: China Merchants Health Industry (Qichun) Co., Ltd., Huanggang City (CN); Tiangong University, Tianjin (CN)

(72) Inventors: Xiaoming QIAN, Tianjin (CN); Jin ZHANG, Tianjin (CN); Yuan ZENG, Huanggang City (CN); Yan FENG, Tianjin (CN); Wenyu WANG, Tianjin (CN); Xing LIU, Tianjin (CN); Yunlong SHI, Tianjin (CN); Yongchao DUO, Tianjin (CN)

(21) Appl. No.: 17/365,036

(22) Filed: Jul. 1, 2021

(30) Foreign Application Priority Data
Jul. 1, 2020 (CN) 202010623000.2

(51) Int. Cl. D06M 16/00; A01N 25/34; A01N 65/12; A01N 65/42; A01N 65/00; A01N 65/08; A01N 65/22; D04H 3/11; D06M 23/10

(52) U.S. Cl. CPC D06M 16/00; A01N 25/34; A01N 65/12; A01N 65/42; D06M 2101/06; A01N 65/08; A01N 65/22; D04H 3/11; D06M 23/10; A01N 65/00

(57) ABSTRACT

A method for making a fiber containing active ingredients from a Chinese herb. The method includes subjecting a fiber material, which is to be combined with active ingredients from the Chinese herb, to be swelled with a solvent of cellulose and then to be impregnated in a solution of an extract of the Chinese herb. The fiber material is a cellulose fiber or a spunlaced nonwoven fabric made thereof. The method requires no use of adhesives. With the method, the fiber material does not need to be subjected to acidic or basic solutions or high temperature, which would cause the active ingredients to be decomposed. The method can provide a cellulose fiber or a nonwoven fabric made thereof, both of which have a higher content of active ingredients from a Chinese herb and higher durability. Furthermore, the method is simple and is easy to control, and thus, suitable for mass production.

### CROSS REFERENCE TO RELATED APPLICATION

The present patent application claims the benefit and priority under 35 U.S.C. § 119 to Chinese Patent Application No. 202010623000.2 (filed on Jul. 1, 2020), the disclosure of which is incorporated by reference herein in its complete entirety as part of the present application.

### TECHNICAL FIELD

The present disclosure is related to the field of fabric production, and, in particular, to a method for making a fiber containing active ingredients from a Chinese herb.

### BACKGROUND

A lot of researchers in China and abroad have proposed the combination of textile fibers with active ingredients from Chinese herbs to provide the textile fibers (and thus the textiles) with antibacterial activities and health functions. Generally, conventional methods for realizing this combination mainly include the following ones. Firstly, active ingredients from Chinese herbs can be introduced into a fiber forming polymer solution or melt via copolymerizing, blending, grafting or the like, which is then spun to produce a fiber material having the same function as that of the active ingredients from Chinese herbs combined therewith (see, for examples, Chinese patent application No. 201510151268.X, titled Artemisia argyi Viscose Fiber and Preparation Method thereof and Chinese patent application No. 201610674286.0, titled Method for Preparing High-strength Artemisia argyi Bacteriostatic Viscose Fiber). The fiber material may, in turn, be used to produce functional textiles via a spinning and weaving process or a nonwoven process. Secondly, Chinese herbal fibers may be blended in the spinning process to produce functional yarns (see, for example, Chinese patent application No. 201410801050.X, titled Wormwood Fiber Composite Textile Material and Preparing Method Thereof). Thirdly, active ingredients from Chinese herbs, to which a finishing agent is added, may be combined into fibers or onto surfaces of textiles by a finishing process such as impregnation or application thereof (see, for example, Chinese patent application No. 201910631241.9, titled Production Technology of Sarcandra glabra Fiber Antibacterial One-way Moisture-conductive Fabric). However, these methods surfer from disadvantages. For example, most of the active ingredients from Chinese herbs are volatile oils, ketones, tannins, and polysaccharides, and they may be decomposed due to the acidic or basic environment or high temperature to which they are subjected in the spinning process, thereby leading to a low content thereof in the fiber products. Or, the active ingredients from Chinese herbs tend to fall off from the textile surfaces during use due to low adhesion therebetween caused by the finishing process. In this case, however, if adhesives are employed to enhance the adhesion of the active ingredients from Chinese herbs to the textile surfaces, the final fabrics would have a stiff feeling, leading to a poor use sensation.

Spunlaced nonwoven fabrics made of cellulose fibers, such as, spunlaced nonwoven fabrics made of viscose or cotton fibers, are widely used in fields including medical protective products and personal care products due to their soft handfeel, hygienic character, and high water absorption capacity. Providing spunlaced nonwoven fabrics with antibacterial effects is an important approach for improving quality and additional value thereof.

### SUMMARY

An objective of the present disclosure is to provide a method for making a fiber containing active ingredients from a Chinese herb.

Accordingly, the objective of the present disclosure is realized by a method for making a fiber containing active ingredients from a Chinese herb, comprising subjecting a fiber material, which is to be combined with active ingredients from a Chinese herb, to be swelled with a solvent of cellulose and then to be impregnated in a solution of an extract of the Chinese herb;

wherein, the fiber material is a cellulose fiber or a spunlaced nonwoven fabric made thereof.

Further, the cellulose fiber may be one of viscose and cotton fibers.

Further, the solvent of cellulose may be a sodium hydroxide (NaOH)/urea or dimethylacetamide (DMAC)/lithium chloride (LiCl) solvent system, an ionic liquid, or an aqueous N-methyl-morpholine-N-oxide (NMMO) solution. Among them, the aqueous NMMO solution is preferred due to its good solubility, easy recovery, and environmental compatibility. Further, the aqueous NMMO solution may have a NMMO concentration of 50 to 70 wt. %. The fiber material may be swelled with the solvent of cellulose at a temperature of 70 to 90 ° C. for 1 to 10 minutes. The swelling degree of the fiber material can be controlled by controlling parameters associated with the swelling process including concentration of the solvent of cellulose, temperature, and time, such that the fiber material swells only at the surface and its internal structure is not destroyed.

Further, the Chinese herb extract solution may have a concentration of 2 to 10 wt. %. In the method as described above, after having been swelled, the fiber material is impregnated in the Chinese herb extract solution so that active ingredients in the Chinese herb extract are adsorbed onto the surface of the fiber material and further dispersed into the inside of the material. The degree of the adsorption and diffusion of the active ingredients with respect to the fiber material can be controlled by controlling parameters associated with the impregnation process including time and concentration of the Chinese herb extract solution. The swelled surface of the fiber material enables an improved adsorption of the active ingredients and provides diffusion channels that lead to the inside of the fiber material.

In an embodiment where the fiber material is a cellulose fiber, the method may further comprise, after the impregnation step, subjecting the cellulose fiber to coagulation via a coagulation bath, washing, and then drying so as to obtain a composite of the cellulose fiber and the Chinese herb extract. The coagulation bath can facilitate in situ fixation of the active ingredients from the Chinese herb, which have been adsorbed on the surface of and in the cellulose fiber, while coagulating the swelled surface of the fiber.

In an embodiment where the fiber material is a spunlaced nonwoven fabric made of cellulose fibers, the method may comprise: impregnating a spunlaced, but not yet dried nonwoven fabric made of cellulose fibers in an aqueous NMMO solution having an appropriate concentration, so that surfaces of the cellulose fibers making up the nonwoven fabric are partly swelled; impregnating the swelled fabric in the Chinese herb extract solution so that active ingredients in the extract are adsorbed onto the surfaces of the cellulose fibers making up the nonwoven fabric and further dispersed into the inside of the cellulose fibers; and subjecting the nonwoven fabric to coagulation via a coagulation bath, washing, and then drying so as to obtain a nonwoven fabric made of the cellulose fibers containing the active ingredients from the Chinese herb. The coagulation bath can facilitate in situ fixation of the active ingredients from the Chinese herb, which have been adsorbed on the surfaces of and in the cellulose fibers, while coagulating the swelled surfaces of the cellulose fibers.

Further, the Chinese herb extract may be selected to have antibacterial activities. Preferably, the Chinese herb is one or more of Artemisia argyi, Aloe vera, Sarcandra glabra, Ionicera japonica, and Mentha canadensis.

The method of the present disclosure has several advantages over the prior art. As is apparent from the above description, the method requires no use of adhesives. Moreover, with the present method, the fiber material needs not be subjected to acidic or basic solutions or high temperature, which would cause active ingredients from a Chinese herb to be decomposed, as mentioned in the background art. Therefore, the present method can provide a cellulose fiber or a nonwoven fabric made of cellulose fibers, both of which have a higher content of active ingredients from a Chinese herb and higher durability. Finally, the method is simple and is easy to control and thus suitable for mass production.

### DETAILED DESCRIPTION

Various terms used hereinafter to describe the examples of the present disclosure will be understood to have the meaning known to persons of ordinary skill in the art, unless otherwise defined. The reagents used in the following examples are all customary ones unless otherwise indicated. Also, experimental processes employed in the examples may be performed in any conventional manners unless otherwise indicated.

### EXAMPLE 1

A viscose fiber (having a white color) was impregnated in an aqueous NMMO solution having a concentration of 70 wt. % at 90° C. for 20 min so that the surface of the viscose fiber was partly swelled. After the fiber was removed from the solution and cooled to room temperature, it was impregnated in an Artemisia argyi extract solution having a concentration of 50 g/L for 5 min. After removal from this solution, the fiber was coagulated via an aqueous coagulation bath at room temperature. Thereafter, the coagulated fiber was washed and then dried. Finally, a composite of the viscose fiber and the Artemisia argyi extract, having a gray green color, was obtained. It was found that the composite obtained had a content of Artemisia argyi extract of 3 wt. %, and exhibited obvious inhibiting effect on Staphylococcus aureus and Escherichia coli, with bacteriostasis rate reaching 93% and 90%, respectively.

### EXAMPLE 2

A viscose fiber (having a white color) was impregnated in an aqueous NMMO solution having a concentration of 60 wt. % at 90° C. for 20 min so that the surface of the viscose fiber was partly swelled. After the fiber was removed from the solution and cooled to room temperature, it was impregnated in an Artemisia argyi extract solution having a concentration of 75 g/L for 5 min. After removal from this solution, the fiber was coagulated via an aqueous coagulation bath at room temperature. Thereafter, the coagulated fiber was washed and then dried. Finally, a composite of the viscose fiber and the Artemisia argyi extract, having a gray green color, was obtained. It was found that the composite obtained had a content of Artemisia argyi extract of 5 wt. %, and exhibited obvious inhibiting effect on Staphylococcus aureus and Escherichia coli, with bacteriostasis rate reaching 95% and 91%, respectively.

### EXAMPLE 3

A cotton fiber (having a white color) was impregnated in an aqueous NMMO solution having a concentration of 60 wt. % at 90° C. for 20 min so that the surface of the cotton fiber was partly swelled. After the fiber was removed from the solution and cooled to room temperature, it was impregnated in an Artemisia argyi extract solution having a concentration of 50 g/L for 5 min. After removal from this solution, the fiber was coagulated via an aqueous coagulation bath at room temperature. Thereafter, the coagulated fiber was washed and then dried. Finally, a composite of the cotton fiber and the Artemisia argyi extract, having a gray green color, was obtained. It was found that the composite obtained had a content of Artemisia argyi extract of 2.5 wt. %, and exhibited obvious inhibiting effect on Staphylococcus aureus and Escherichia coli, with bacteriostasis rate reaching 90% and 85%, respectively.

### EXAMPLE 4

A viscose fiber (having a white color) was impregnated in an aqueous NMMO solution having a concentration of 50 wt. % at 70° C. for 20 min so that the surface of the viscose fiber was partly swelled. After the fiber was removed from the solution and cooled to room temperature, it was impregnated in an Artemisia argyi extract solution having a concentration of 50 g/L for 8 min. After removal from this solution, the fiber was coagulated via an aqueous coagulation bath at room temperature. Thereafter, the coagulated fiber was washed and then dried. Finally, a composite of the viscose fiber and the Artemisia argyi extract, having a gray green color, was obtained. It was found that the composite obtained had a content of Artemisia argyi extract of 2.2 wt. %, and exhibited obvious inhibiting effect on Staphylococcus aureus and Escherichia coli, with bacteriostasis rate reaching 88% and 80%, respectively.

### EXAMPLE 5

A viscose fiber (having a white color) was impregnated in an aqueous NMMO solution having a concentration of 60 wt. % at 90° C. for 40 min so that the surface of the viscose fiber was partly swelled. After the fiber was removed from the solution and cooled to room temperature, it was impregnated in an Artemisia argyi extract solution having a concentration of 50 g/L for 5 min. After removal from this solution, the fiber was coagulated via an aqueous coagulation bath at room temperature. Thereafter, the coagulated fiber was washed and then dried. Finally, a composite of the viscose fiber and the Artemisia argyi extract, having a gray green color, was obtained. It was found that the composite obtained had a content of Artemisia argyi extract of 5.5 wt. %, and exhibited obvious inhibiting effect on Staphylococcus aureus and Escherichia coli, with bacteriostasis rate reaching 95% and 91%, respectively.

### EXAMPLE 6

A viscose fiber was impregnated in an aqueous NMMO solution having a concentration of 60 wt. % at 90° C. for 20 min so that the surface of the viscose fiber was partly swelled. After the fiber was removed from the solution and cooled to room temperature, it was impregnated in a Sarcandra glabra extract solution having a concentration of 75 g/L for 5 min. After removal from this solution, the fiber was coagulated via an aqueous coagulation bath at room temperature. Thereafter, the coagulated fiber was washed and then dried. Finally, a composite of the viscose fiber and the Sarcandra glabra extract was obtained. It was found that the composite obtained had a content of Sarcandra glabra extract of 4.5 wt. %, and exhibited obvious inhibiting effect on Staphylococcus aureus and Escherichia coli, with bacteriostasis rate reaching 90% and 87%, respectively.

### EXAMPLE 7

A viscose fiber was impregnated in an aqueous NMMO solution having a concentration of 60 wt. % at 90° C. for 20 min so that the surface of the viscose fiber was partly swelled. After the fiber was removed from the solution and cooled to room temperature, it was impregnated in an Aloe vera extract solution having a concentration of 75 g/L for 5 min. After removal from this solution, the fiber was coagulated via an aqueous coagulation bath at room temperature. Thereafter, the coagulated fiber was washed and then dried. Finally, a composite of the viscose fiber and the Aloe vera extract was obtained. It was found that the composite obtained had a content of Aloe vera extract of 4 wt. %, and exhibited obvious inhibiting effect on Staphylococcus aureus and Escherichia coli, with bacteriostasis rate reaching 89% and 84%, respectively.

### EXAMPLE 8

A spunlaced, but not yet dried nonwoven fabric made of cellulose fibers was impregnated in an aqueous NMMO solution having a concentration of 70 wt. % at 90° C. for 20 min so that surfaces of the cellulose fibers of the nonwoven fabric were partly swelled. After the fabric was removed from the solution and cooled to room temperature, it was impregnated in an Artemisia argyi extract solution having a concentration of 75 g/L for 5 min. After removal from this solution, the fabric was coagulated via an aqueous coagulation bath at room temperature. Thereafter, the coagulated fabric was washed and then dried. Finally, a composite of the fabric and the Artemisia argyi extract was obtained. It was found that the composite obtained had a content of Artemisia argyi extract of 3 wt. %, and exhibited obvious inhibiting effect on Staphylococcus aureus and Escherichia coli, with bacteriostasis rate reaching 90% and 86%, respectively.

Although the present disclosure has been described above with respect to particular embodiments, the scope of the disclosure is not limited to the particular embodiments described above. Furthermore, the embodiments should be considered to be illustrative and not limiting. It will be apparent to persons of ordinary skill in the art that various modifications and variations may be made without departing from the scope of the present disclosure.

What is claimed is:

1. A method for making a fiber containing active ingredients from a Chinese herb, the method comprising:
subjecting a fiber material, which is to be combined with active ingredients from the Chinese herb, to be swelled with a solvent of cellulose and then to be impregnated in a solution of an extract of the Chinese herb,
wherein, the fiber material is a cellulose fiber or a spunlaced nonwoven fabric made thereof.

2. The method of claim 1, wherein the cellulose fiber is one of viscose fibers and cotton fibers.

3. The method of claim 1, wherein the solvent of cellulose is an aqueous N-methyl-morpholine-N-oxide solution having a concentration of 50 to 70 wt. %.

4. The method of claim 1, wherein the fiber material is swelled with the solvent of cellulose for 1 to 10 minutes.

5. The method of claim 1, wherein the fiber material is swelled with the solvent of cellulose at a temperature of 70 to 90° C.

6. The method of claim 1, wherein the Chinese herb extract solution has a concentration of 2 to 10 wt. %.

7. The method of claim 1, further comprising, when the fiber material is a cellulose fiber:
after the impregnation, subjecting the cellulose fiber to coagulation via a coagulation bath, washing, and then drying so as to obtain a composite of the cellulose fiber and the Chinese herb extract.

8. The method of claim 1, further comprising, when the fiber material is a spunlaced nonwoven fabric made of cellulose fibers:
after the impregnation, subjecting the nonwoven fabric to coagulation via a coagulation bath, washing, and then drying so as to obtain a composite of the nonwoven fabric and the Chinese herb extract.

9. The method of claim 1, wherein the Chinese herb extract is selected to have antibacterial activities.

10. The method of claim 1, wherein the Chinese herb is one or more of Artemisia argyi, Aloe vera, Sarcandra glabra, Ionicera japonica, and Mentha canadensis.
